(19) United States
(12) Patent Application Publication
(10) Pub. No.: US 2015/0151966 A1
(43) Pub. Date: Jun. 4, 2015

(54) SEPARATION METHOD OF TELLURIUM AND SELENIUM, AND PREPARATION METHOD OF TELLURIUM USING THE SAME

(71) Applicants: KOREA INSTITUTE OF SCIENCE AND TECHNOLOGY, Seoul (KR); RECYTEC INC., Namyangju-si (KR)

(72) Inventors: Hwa Young LEE, Seoul (KR); Joong Kee LEE, Seoul (KR); Jae Gyu JEE, Seongnam-si (KR); Joon Chul CHOI, Gunpo-si (KR)

(21) Appl. No.: 14/541,799

(22) Filed: Nov. 14, 2014

(30) Foreign Application Priority Data
Dec. 3, 2013 (KR) 10-2013-0149460

(51) Int. Cl. C01B 19/00; C07C 51/42

(52) U.S. Cl. CPC C01B 19/001; C07C 51/42

(57) ABSTRACT

The present invention relates to a method of separating tellurium (Te) and selenium (Se) by using a neutral extractant (for example, either one of TBP and TEHP, or in combination thereof) from a dissolved solution of tellurium, such as a waste thermoelectric material. When tellurium and selenium are separated by using the method of this invention from a dissolved solution in which a Bi₂Te₃-based waste thermoelectric material is dissolved, a recovery rate of 97% or more and a separation factor of 400 or more for tellurium may be achieved, and therefore, tellurium and selenium may be separated in a very effective and economic manner as compared to conventional methods. Further, the present invention is characterized in that environmental pollution issues may be significantly reduced as compared with conventional methods carried out in a strong acidic solution since the present separation and extraction is conducted under a relative weak acid atmosphere in a pH range of 1.0 to 1.5.

### CROSS-REFERENCE TO RELATED APPLICATION

Pursuant to 35 U.S.C. §119(a), this application claims the benefit of earlier filing date and right of priority to Korean Application No. 10-2013-0149460, filed on Dec. 3, 2013, the contents of which is incorporated by reference herein in its entirety.

### BACKGROUND OF THE DISCLOSURE

1. Field of the Disclosure

The present disclosure relates to a separation method of tellurium (Te) and selenium (Se) and a method of preparing tellurium recovered with a high recovery rate while removing selenium, and particularly, to a method of effectively separating tellurium and selenium from a dissolved solution of a material containing tellurium, such as a waste thermoelectric material, by mixing the solution with a neutral extractant or a mixture thereof, and preparing high-purity tellurium using the same.

2. Background of the Disclosure

Recently, various electronic products or refrigeration products using thermoelectric materials have been widely supplied. The thermoelectric material is a kind of semiconductor material having a function of taking electrical energy and using it to transfer heat, or conversely taking heat and converting it into electrical energy.

Such thermoelectric materials have been applied to electric power generation which uses waste heat, or a small refrigeration product such as a hot/cold water service machine. Further, a vehicle cooling sheet has lately been equipped with a thermoelectric material module, thereby providing a driver with a comfortable environment in a hot and humid summer season.

Among the thermoelectric materials, Bi₂Te₃ is a material which has been currently utilized in the most common manner, and the thermoelectric materials have been known to have a lifespan of about 10 years. However, tellurium (Te), which is a main component among the components which constitute the Bi₂Te₃ thermoelectric material, is one of very rare metals, and its natural abundance on earth is still scarce as well as the amount of the element produced in the world per year is estimated at only several hundred tons. Therefore, it is necessary to separate and recycle tellurium from a waste thermoelectric material that has been used up to the limit of its lifespan.

In general, the thermoelectric material module contains a trace of metal such as Sb and Se due to various additives other than Bi₂Te₃ which is a key material, and it is certainly necessary to separate/recover tellurium from these metals for the aforementioned tellurium recycling.

Among the metals, selenium (Se) is a problematic metal particularly in separating tellurium. Since physicochemical properties of tellurium and selenium are very similar to each other, it has been known to be highly difficult to separate the two metals using a conventional separation method.

In the separation of tellurium and selenium, examples of a separation method that has been reported until now include a solvent separation method using various extractants, a neutralization precipitation method through pH adjustment, an ion exchange method, and so forth.

Among them, the neutralization precipitation method has a problem in that the effect of separating tellurium and selenium is not so significant, and the ion exchange method needs to use an expensive ion exchange resin, resulting in an economic burden.

In addition, in separation of tellurium and selenium using a solvent extraction method, the two metals are separated by adjusting the concentration of hydrochloric acid under a strong acidic atmosphere with a hydrochloric acid concentration in a range of 2 to 6 mole, but in this case, since the difference in extraction rates between the two metals is not very great depending on the hydrochloric acid concentration, a satisfactory separation effect may not be obtainable by performing only a single step operation, and these metals need to be separated by performing a multi-step operation. Therefore, when tellurium and selenium are separated using the solvent extraction method, there is a problem in that process cost is excessively increased due to the multi-step operation along with environmental pollution issues resulting from the use of strong acidic solutions.

The present invention is intended to provide a solvent extraction method for separating tellurium and selenium in a very effective and economic manner, which are contained in the dissolved solution obtained by dissolving a Bi₂Te₃-based waste thermoelectric module, thereby contributing to the recovery and recycling of high-purity tellurium from the waste thermoelectric material.

### CITATION LIST

### Patent Document

(Patent Document 0001) 1. U.S. Pat. No. 5,939,042, Tellurium extraction from copper electrofefining slimes

### SUMMARY OF THE DISCLOSURE

Therefore, an aspect of the detailed description is to provide a method for recovering and recycling tellurium which is an expensive rare metal from a material including both Te and Se, such as a Bi₂Te₃-based waste thermoelectric material. Specifically, an aspect of the detailed description is to provide a solvent extraction method for effectively separating tellurium and selenium from a dissolved solution of the material containing Te by using a neutral extractant, and a method of preparing high-purity tellurium by applying such extraction method.

To achieve these and other advantages and in accordance with the purpose of this specification, as embodied and broadly described herein, a separation method of tellurium (Te) and selenium (Se) according to one embodiment of the present invention includes the steps of: preparing a dissolved solution of a material comprising tellurium (Te) and selenium (Se) by dissolving the material in a solution containing a strong acid (1); preparing a pre-treatment solution by adding a base to the dissolved solution to adjust a pH of the dissolved solution to a range of 1.0 to 1.5 (2); allowing the pre-treatment solution to contain oxalate ions (C₂O₄²⁻) and preparing a complex compound solution in which a complex compound is formed by reacting tellurium ions and selenium ions in the pre-treatment solution with the oxalate ions, respectively (3); and obtaining a first extraction solution as an organic phase prepared by using a solvent extraction method which uses a neutral extractant to selectively transfer a tellurium oxalate complex compound in the complex compound solution as an aqueous solution phase to the neutral extractant as an organic phase (4).

The strong acid in step (1) may be any one selected from the group consisting of nitric acid, hydrochloric acid and a combination thereof.

In step (2), the dissolved solution may include chlorine ions, and a precipitation is produced during a process that the pH of the dissolved solution to which the base is added is adjusted to the range, so that the pre-treatment solution may be prepared by removing the precipitation.

The oxalate ions may be derived from oxalic acid (H₂C₂O₄).

The oxalate-ions in step (3) may be in an amount of 0.05 to 0.3 mole per one liter of the pre-treatment solution.

The neutral extractant in step (4) may include any one selected from the group consisting of tributyl phosphate (TBP), tris(2-ethylhexyl) phosphate (TEHP) and a combination thereof.

Tellurium recovered from the first extraction solution in step (4) may have a recovery rate of 97% or more and a separation factor of 400 or more.

The material containing tellurium (Te) and selenium (Se) may include a Bi₂Te₃-based waste thermoelectric material.

A preparation method of tellurium (Te) separated from selenium (Se) according to another embodiment of the present invention includes the steps of: preparing a dissolved solution of a material comprising tellurium (Te) and selenium (Se) by dissolving the material in a solution containing a strong acid (1); preparing a pre-treatment solution by adding a base to the dissolved solution to adjust a pH of the dissolved solution to a range from 1.0 to 1.5 (2); allowing the pre-treatment solution to contain oxalate ions (C₂O₄²⁻) and preparing a complex compound solution in which a complex compound is formed by reacting tellurium ions and selenium ions in the pre-treatment solution with the oxalate ions, respectively (3); obtaining a first extraction solution as an organic phase prepared by using a solvent extraction method which uses a neutral extractant to selectively transfer a tellurium oxalate complex compound in the complex compound solution as an aqueous solution phase to the neutral extractant as an organic phase (4); and preparing a second extraction solution as an aqueous solution phase, which comprises tellurium ions prepared by stirring with an acid aqueous solution for reverse extraction the first extraction solution which is phase-separated to transfer tellurium included in the first extraction solution as an organic phase to the acid aqueous solution (5).

The acid aqueous solution in step (5) may be a weak acidic aqueous solution, and may include hydrochloric acid in an amount of 0.2 to 0.5 mole/L.

Hereinafter, the present invention will be described in more detail.

In the present invention, reference to a material containing tellurium (Te) is and selenium (Se) refers to a material for separating tellurium (Te) from other metals including selenium (Se), and an example thereof includes a Bi₂Te₃-based waste thermoelectric material, but is not limited to.

The present invention provides a separation method of tellurium (Te) and selenium (Se) according to an exemplary embodiment of this invention, the method comprising the steps of: preparing a dissolved solution of a material including tellurium (Te) and selenium (Se) by dissolving the material in a solution containing a strong acid (1); preparing a pre-treatment solution by adding a base to the dissolved solution to adjust a pH of the dissolved solution to a range of 1.0 to 1.5 (2); allowing the pre-treatment solution to contain oxalate ions (C₂O₄²⁻) and preparing a complex compound solution in which a complex compound is formed by reacting tellurium ions and selenium ions in the pre-treatment solution with the oxalate ions, respectively (3); and obtaining a first extraction solution as an organic phase prepared by using a solvent extraction method which uses a neutral extractant to selectively transfer a tellurium oxalate complex compound in the complex compound solution as an aqueous solution phase to the neutral extractant as an organic phase (4), thereby effectively separating tellurium and selenium.

The strong acid in step (1) refers to a strong acid having a pH less than 1. Where a material containing tellurium and selenium (for example, a waste thermoelectric material) is ground to an appropriate size and then dissolved in the strong acid, a dissolved solution is prepared, in which metals are dissolved in the form of metal ions in a strong acidic solution. The solution containing the strong acid may be a strong hydrochloric acid or strong nitric acid aqueous solution, and may be a mixture thereof.

The base in step (2) may be applied as long as it adjusts the pH of the dissolved solution and does not disturb the subsequent reactions, and may be, for example, any one selected from the group consisting of sodium hydroxide, potassium hydroxide, ammonium hydroxide and a combination thereof.

The pH of the dissolved solution to which the base is added is set to be in a range of 1.0 to 1.5. When the pH of the dissolved solution to which the base is added is less than 1.0, the recovery rate of tellurium may be lowered, and when the pH is more than 1.5, tellurium and selenium are simultaneously extracted, so that a separation effect of these metals may deteriorate.

Furthermore, when hydrochloric acid as the strong acid is applied to the dissolved solution adjusted to the pH range, or chlorine ions are included in the dissolved solution, some elements such as Sb and Bi contained in the dissolved solution may be precipitated to produce an insoluble precipitate such as antimony oxychloride (SbOCl) and the like, and these elements may be removed by a simple process of filtering the precipitate. Meanwhile, tellurium and selenium in the dissolved solution are not precipitated in the pH range, and thus are remaining in the form of ions as they are in the dissolved solution even when the pH of the dissolved solution may be adjusted within the aforementioned range.

The step (3) is a process of forming a complex compound by adding oxalate ions to the pre-treatment solution, and allows tellurium ions and selenium ions in the pre-treatment solution to be bonded to oxalate ions (C₂O₄²⁻), respectively, thereby forming the complex compound. In this case, there is a significant difference in affinity for a neutral extractant (for example, TBP, TEHP) between a tellurium-oxalate complex compound and a selenium-oxalate complex compound to be produced according to the pH of the solution. Accordingly, the present method makes it possible to separately extract tellurium and selenium, which would be difficult to mutually separate because the two metals have similar properties.

For the oxalate ions, the complex compound solution may be prepared by a process of adding oxalic acid (H₂C₂O₄) to the pre-treatment solution.

The oxalic acid may be added to the pre-treatment solution, such that oxalic acid ions are formed in an amount of 0.05 to 0.3 mole per one liter of the pre-treatment solution. When the content of oxalic acid ions in the pre-treatment solution is smaller than the range, the separation effect of tellurium and selenium may deteriorate, and when the content exceeds the range, the amount of an agent consumed may be unnecessarily increased.

The step (4) is a step to selectively extract tellurium from the pre-treatment solution using a solvent extraction method with a neutral extractant. Specifically, since tellurium present in a complex compound solution as an aqueous solution phase is extracted by a neutral extractant as an organic phase, and in this case, only tellurium (in the form of a complex compound) is selectively extracted into the neutral extractant, it may be separated from selenium.

The neutral extractant may include any one selected from the group consisting of tributyl phosphate (TBP), tris(2-ethylhexyl) phosphate (TEHP) and a combination thereof. When TBP or TEHP is contained, or a mixture thereof is applied as the neutral extractant, tellurium may be selectively extracted with a high recovery rate. In this case, selenium is still remaining as it is in the complex compound solution.

In particular, when the above-described separation method is performed by applying a base by mean of the strong acid or allowing chlorine ions to be present in the pre-treatment solution, and including at least one of TBP and TEHP as the neutral extractant, the recovery rate of tellurium may be enhanced to 97% or more, and the separation factor of tellurium and selenium may be greatly increased to 400 or more.

The neutral extractant further includes a diluent, and the diluent may be any one selected from the group consisting of kerosene, hexane, benzene, toluene and a combination thereof.

The amount of the neutral extractant used is not particularly limited, but may be, for example, 0.5 to 2 times by volume more than that of the complex compound solution, and the neutral extractant and the complex compound solution may be utilized in a volume ratio of about 1:1.

The separation method of tellurium (Te) and selenium (Se) may further include step (5) of reverse extracting tellurium after the step (4).

The step (5) is a step of preparing a second extraction solution as an aqueous solution phase which contains tellurium in the form of ions by stirring with an acid aqueous solution for reverse extraction the first extraction solution which is phase-separated to transfer tellurium contained in the first extraction solution as an organic phase to the acid aqueous solution.

The acid aqueous solution for reverse extraction may be a weak acidic aqueous solution, and a weak hydrochloric acid aqueous solution. The acid aqueous solution may include hydrochloric acid at a concentration of 0.2 to 0.5 mole/L. When the range of the concentration is less than 0.2 mole/L, tellurium may not be sufficiently reverse extracted, and when the range exceeds 0.5 mole/L, an agent may be unnecessarily consumed.

The preparation method of tellurium (Te) separated from selenium (Se) according to another exemplary embodiment of the present invention provides a method of preparing tellurium having a very small content of selenium by separating tellurium and selenium, which would be difficult to mutually separate at high recovery rate and separation factor. Since each step, agent to be used, reactions in each step and the like of the preparation method of tellurium (Te) separated from selenium (Se) are overlapped with those described above, the description thereof will be omitted.

The present invention provides a method for recovering and recycling Te which is an expensive rare metal from a dissolved solution in which a material such as a Bi₂Te₃-based waste thermoelectric material is dissolved (preferably, dissolved in a hydrochloric acid or nitric acid aqueous solution).

A complex compound of tellurium and selenium in a dissolved solution is formed by adding a base such as caustic soda (NaOH) to the dissolved solution in which a material such as a waste thermoelectric material is dissolved using a strong acid to adjust the pH of the dissolved solution to a range of 1.0 to 1.5, and then adding a compound which provides oxalate ions such as oxalic acid (H₂C₂O₄).

Since the complex compounds produced by bonding oxalate (C₂O₄²⁻) ions to tellurium or selenium have a relatively large difference in physicochemical properties, an effect of extracting and separating the solvent may be greatly enhanced by the neutral extractant such as TBP or TEHP.

In particular, it is common that metal extraction using TBP or TEHP as a neutral extractant is mostly carried out under a strong acidic atmosphere, but the method of the present invention is remarkably characterized in that tellurium and selenium are separated and extracted under a weak acid atmosphere in a pH range of 1.0 to 1.5, as described above.

Further, both the separation method of tellurium (Te) and selenium (Se) and the preparation method of tellurium (Te) separated from selenium (Se) are processes capable of being performed at room temperature (15 to 25° C.), and thus are economically advantageous as compared to methods which essentially require processing at high temperature.

The separation method of tellurium (Te) and selenium (Se) and the preparation method of tellurium (Te) separated from selenium (Se) of the present invention may effectively separate these metals from a dissolved solution in which a material containing both tellurium (Te) and selenium (Se), such as a Bi₂Te₃-based waste thermoelectric material, is dissolved. In particular, the methods may achieve a tellurium recovery rate of 97% or more and a separation factor (distribution coefficient ratio of metals to be separated) of 400 or more, and thus may separate tellurium and selenium in a very effective and economic manner as compared with conventional methods. In addition, since the separation and extraction is performed under a relative weak acid atmosphere in a pH range of 1.0 to 1.5, the methods are advantageous in that environmental pollution issues may be significantly reduced as compared to conventional methods which have utilized a strong acidic solution.

Further scope of applicability of the present application will become more apparent from the detailed description given hereinafter. However, it should be understood that the detailed description and specific examples, while indicating preferred embodiments of the disclosure, are given by way of illustration only, since various changes and modifications within the spirit and scope of the disclosure will become apparent to those skilled in the art from the detailed description.

### DETAILED DESCRIPTION OF THE DISCLOSURE

Hereinafter, exemplary embodiments of the present invention will be described in detail for a person ordinarily skilled in the art to easily carry out. However, the present invention may be implemented in various forms, and is not limited to the exemplary embodiments described herein.

### EXAMPLE 1

Step (1): A Bi₂Te₃-based waste thermoelectric material separated from a waste vehicle sheet was ground to an appropriate size, and dissolved the material in a hydrochloric acid aqueous solution which is a strong acid, to prepare a dissolved solution (dissolved solution of the waste thermoelectric material). In this case, each content of tellurium and selenium in the dissolved solution was found to be 5.350 mg/l and 127 mg/l.

Step (2): A pre-treatment solution was prepared by adding caustic soda to the dissolved solution, and adjusting the pH of the dissolved solution to be 1.5, followed by filtering the precipitate.

Step (3): A complex compound solution was prepared by adding oxalic acid to the pre-treatment solution such that the concentration thereof was 0.05 mole/l.

Step (4): One liter of tributyl phosphate (TBP) as a neutral extractant and one liter of the complex compound solution were put into a reactor, and the mixture was vigorously stirred at room temperature for 1 hour. Then, a first extraction solution of an organic phase was obtained by selectively extracting tellurium in the complex compound solution using a neutral extractant as an organic phase.

After the solvent extraction process in step (4) was completed, the organic phase and the aqueous solution phase were subjected to a phase separation to obtain tellurium and selenium of an aqueous solution phase (dissolved solution subjected to the solvent extraction process), and the content thereof was analyzed by using ICP Spectrophotometer (Perkin Elmer, Model: Optima 5300 DV). As a result, each content of tellurium and selenium was shown to be 125 mg/l and 118 mg/l.

Step (5): One liter of the first extraction solution as a loaded organic phase obtained during the extraction process and one liter of a weak acidic aqueous solution (an acidic aqueous solution for reverse extraction) having a hydrochloric acid concentration of 0.2 mole/l were put into a reactor, and the mixture was vigorously stirred at room temperature for 1 hour. Then, tellurium was recovered by transferring tellurium contained in the first extraction solution to the weak acid aqueous solution to prepare a second extraction solution as an aqueous solution phase.

After step (5) including the reverse extraction process was completed, the content of tellurium and selenium in an aqueous solution obtained by performing a phase separation was analyzed. As a result, the content thereof was found to be 5.220 mg/l and 6.5 mg/l, respectively.

Accordingly, the final recovery rate of tellurium was 97.6% (=5.220/5.350) based on tellurium in the initially dissolved solution, each distribution coefficient of tellurium and selenium was shown to be 41.8 {=(5.350−125)/125} and 0.076 {=(127−118)/118}, and consequently, the separation factor of tellurium and selenium was shown to be 550 (=41.8/0.076).

### EXAMPLE 2

Steps (1) and (2): A pre-treatment solution was prepared by adding caustic soda to the dissolved solution of the waste thermoelectric material, which was the same as that in Example 1 to adjust the pH of the dissolved solution to be 1.0, and then filtering the precipitate.

Step (3): A complex compound solution was prepared by adding oxalic acid to the pre-treatment solution such that the concentration thereof was 0.3 mole/l.

Step (4): One liter of tris(2-ethylhexyl) phosphate (TEHP) and one liter of the complex compound solution were put into a reactor, and a first extraction solution as an organic phase was obtained by vigorously stirring the mixture at room temperature for 1 hour in the same manner as in Example 1 to selectively extract tellurium using a neutral extractant as an organic phase.

After the solvent extraction process in step (4) was completed, the organic phase and the aqueous solution phase were subjected to a phase separation to obtain tellurium and selenium as an aqueous solution phase, and the content of tellurium and selenium was analyzed by using ICP Spectrophotometer (Perkin Elmer, Model: Optima 5300 DV). As a result, the content of tellurium and selenium was shown to be 117 mg/l and 115 mg/l, respectively.

Step (5): One liter of the first extraction solution as a loaded organic phase obtained during the extraction process and one liter of a weak acidic aqueous solution (an acidic aqueous solution for reverse extraction) having a hydrochloric acid concentration of 0.5 mole/l were put into a reactor, and tellurium was reverse extracted and recovered into a second extraction solution while vigorously stirring the mixture at room temperature for 1 hour in the same manner as in Example 1.

After the reverse extraction process was completed, the content of tellurium and selenium in an aqueous solution (the second extraction solution) obtained by performing the phase separation was analyzed, and as a result, the content of tellurium and selenium was shown to be 5.230 mg/l and 8.7 mg/l, respectively.

Accordingly, the final recovery rate of tellurium was 97.8% (=5.230/5.350) based on tellurium in the initially dissolved solution, the distribution coefficient of tellurium and selenium was shown to be 44.7 {=(5.350−117)/117} and 0.1 {=(127−115)/115}, respectively, and therefore, the separation factor of tellurium and selenium was found to be 447 (=44.7/0.1).

### EXAMPLE 3

Steps (1) to (3): A pre-treatment solution was prepared by adding caustic soda to the dissolved solution of the waste thermoelectric material, which was the same as that in Example 1 to adjust the pH of the dissolved solution to be 1.5, and then filtering the precipitate. A complex compound solution was prepared by adding oxalic acid to the pre-treatment solution such that the concentration thereof was 0.3 mole/l.

Step (4): One liter of an organic phase obtained by mixing a neutral extractant containing 0.3 liter of TBP and 0.3 liter of TEHP with 0.4 liter of kerosene (a diluent), and one liter of the complex compound solution were put into a reactor, and a first extraction solution as an organic phase was obtained by extracting tellurium into the organic phase while vigorously stirring the mixture at room temperature for 1 hour in the same manner as in Example.

After the solvent extraction process in step (4) was completed, the organic phase and the aqueous solution phase were subjected to a phase separation to obtain tellurium and selenium as an aqueous solution phase, and the content of tellurium and selenium was analyzed by using ICP Spectrophotometer (Perkin Elmer, Model: Optima 5300 DV). As a result, the content of tellurium and selenium was shown to be 128 mg/l and 120 mg/l, respectively.

Step (5): One liter of the first extraction solution as a loaded organic phase obtained during the extraction process and one liter of a weak acidic aqueous solution (an acidic aqueous solution for reverse extraction) having a hydrochloric acid concentration of 0.2 mole/l were put into a reactor, and tellurium was reverse extracted and recovered into a second extraction solution while vigorously stirring the mixture at room temperature for 1 hour in the same manner as in Example 1.

After the reverse extraction process was completed, the content of tellurium and selenium in an aqueous solution (the second extraction solution) obtained by performing the phase separation was analyzed, and as a result, each content of tellurium and selenium was shown to be 5.210 mg/l and 5.3 mg/l.

Accordingly, the final recovery rate of tellurium was 97.4% (=5.210/5.350) based on tellurium in the initially dissolved solution, the distribution coefficient of tellurium and selenium was shown to be 40.8 {=(5.350−128)/128} and 0.058 {=(127−120)/120}, respectively, and in this regard, the separation factor of tellurium and selenium was shown to be 703 (=40.8/0.058).

While preferred embodiments of the present invention have been described in detail, it is to be understood that the scope of the present invention is not limited thereto, and various modifications and variations made by those skilled in the art using basic concepts of the present invention defined in the following claims also fall within the scope of the present invention.

As the present features may be embodied in several forms without departing from the characteristics thereof, it should also be understood that the above-described embodiments are not limited by any of the details of the foregoing description, unless otherwise specified, but rather should be considered broadly within its scope as defined in the appended claims, and therefore all changes and modifications that fall within the metes and bounds of the claims, or equivalents of such metes and bounds are therefore intended to be embraced by the appended claims.

What is claimed is:

1. A separation method of tellurium (Te) and selenium (Se), comprising the steps of:
preparing a dissolved solution of a material comprising tellurium (Te) and selenium (Se) by dissolving the material in a solution comprising a strong acid (1);
preparing a pre-treatment solution by adding a base to the dissolved solution to adjust a pH of the dissolved solution to a range of 1.0 to 1.5 (2);
allowing the pre-treatment solution to comprise oxalate ions (C₂O₄²⁻), and preparing a complex compound solution in which a complex compound is formed by reacting tellurium ions and selenium ions in the pre-treatment solution with the oxalate ions, respectively (3); and
obtaining a first extraction solution as an organic phase prepared by a solvent extraction method which uses a neutral extractant to selectively transfer a tellurium oxalate complex compound in the complex compound solution as an aqueous solution phase to the neutral extractant as an organic phase (4).

2. The method of claim 1, wherein the strong acid in step (1) is any one selected from the group consisting of nitric acid, hydrochloric acid and a combination thereof.

3. The method of claim 1, wherein the dissolved solution in step (2) comprises chlorine ions, a precipitate is produced during a process of adjusting to the range a pH of the dissolved solution to which the base is added, and the pre-treatment solution is prepared by removing the precipitate.

4. The method of claim 1, wherein the oxalate ions are derived from oxalic acid (H₂C₂O₄).

5. The method of claim 1, wherein the oxalate ions in step (3) are in an amount of 0.05 to 0.3 mole per one liter of the pretreatment solution.

6. The method of claim 1, wherein the neutral extractant in step (4) comprises any one selected from the group consisting of tributyl phosphate (TBP), tris(2-ethylhexyl) phosphate (TEHP) and a combination thereof.

7. The method of claim 1, wherein tellurium recovered from the first extraction solution in step (4) has a recovery rate of 97% or more and a separation factor of 400 or more.

8. The method of claim 1, wherein the material comprising tellurium (Te) and selenium (Se) comprises a Bi₂Te₃-based waste thermoelectric material.

9. A preparation method of tellurium (Te) separated from selenium (Se), comprising the steps of:
preparing a dissolved solution of a material comprising tellurium (Te) and selenium (Se) by dissolving the material in a solution comprising a strong acid (1);
preparing a pre-treatment solution by adding a base to the dissolved solution to adjust a pH of the dissolved solution to a range of 1.0 to 1.5 (2);
allowing the pre-treatment solution to comprise oxalate ions (C₂O₄²⁻) and preparing a complex compound solution in which a complex compound is formed by reacting tellurium ions and selenium ions in the pre-treatment solution with the oxalate ions, respectively (3);
obtaining a first extraction solution as an organic phase prepared by a solvent extraction method which uses a neutral extractant to selectively transfer a tellurium oxalate complex compound in the complex compound solution as an aqueous solution phase to the neutral extractant as an organic phase (4); and
preparing a second extraction solution as an aqueous solution phase, which comprises tellurium ions prepared by stirring with an acid aqueous solution for reverse extraction the first extraction solution which is phase-separated to transfer tellurium contained in the first extraction solution as an organic phase to the acid aqueous solution (5).

10. The method of claim 9, wherein the acid aqueous solution in step (5) is a weak acid aqueous solution comprising hydrochloric acid in an amount of 0.2 to 0.5 mole/L.
